(19) United States
(12) Patent Application Publication
(10) Pub. No.: US 2008/0234727 A1
(43) Pub. Date: Sep. 25, 2008

(54) Novel Carriers For Coating Growth Factors Onto Sutures

(76) Inventors: Venkat Garigapati, Southborough, MA (US); Rehan Khanzada, Middleborough, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/689,757

(22) Filed: Mar. 22, 2007

(51) Int. Cl. A61B 17/04

(52) U.S. Cl. 606/228

(57) ABSTRACT

A suture coated with a composition comprising:
a) a growth factor,
b) a biodegradable, non-polymeric, non-water soluble, liquid carrier material (such as SAIB),
c) a growth factor stabilizer,
d) a solvent in which both the biodegradable, non-polymeric, non-water soluble, liquid carrier material and the protein stabilizer are miscible,
e) a volatile alcohol (such as ethanol).

### BACKGROUND OF THE INVENTION

The tissue repair literature has reported the use of growth factor-coated sutures, and particularly describes the use of BMPs such as rhGDF-5 for their ability to form tendon, cartilage, bone and ligament-like structures. For example, Rickert et al., Growth Factors, 19, 2001, 115-126, discloses the use of rhGDF-5 upon sutures to stimulate tendon healing in an Achilles tendon model in rats. See also, U.S. Pat. No. 5,658,882 (Celeste); U.S. Pat. No. 6,187,742 (Wozney); U.S. Pat. No. 6,284,872 (Celeste TI); U.S. Pat. No. 6,719,968 (Celeste ITT); and US Published Patent Application No. 2004/0146923 (Celeste IV).

Coated sutures and implants (with collagen, butyric acid and a variety of growth factors) have been used in soft tissue repair. See, for example, Mazzocca, AAOS Abstract #338, 2005; Wright, 50^{th} ORS, #1234, 2004; Petersen, 51^{st} ORS, #0076, 2005; Schmidmaier, J. Biomedical Materials Res (Appl Biomat) 58, 449-455, 2001. These papers report promising in vitro and in vivo data. However, implantation of these implants into humans using these techniques is not currently possible, as in vitro models require further development and additional data are required to better characterize the in vivo models.

Wright, supra, reports the use of butyric acid treated silicon coated sutures in bilateral meniscal tears in an in vivo sheep model. Wright reports that tears repaired with coated sutures possessed new and repaired tissue including neo-angiogenesis at the repair site. This study demonstrates the potential for connective tissue repair.

Petersen, supra, reports the use of an in vivo sheep model, wherein local application of VEGF using poly(D,L lactide)-sutures stimulated proliferation of blood vessels but did not show enhanced meniscus healing.

Sutures coated with antimicrobials are commercially available for clinical use. At present, polyglactin sutures coated with antibiotics sold under the tradename Coated VICRYL PLUS (polyglactin 910) Suture (Ethicon, Somerville, N.J.) is the first and only antibacterial suture approved by the FDA for inhibiting the colonization of bacteria, which causes the majority of surgical site infections. The VICRYL PLUS suture creates an inhibitory zone around the suture in which bacteria are prevented from making colonies. See Rothenberger, Surgical Infection Society Journal (Supp) Dec. 2002, pp. 579-87, and Mangram, Infection control and Hospital Epidemiology, 1999, 20(40, 247-280. The VICRYL PLUS suture contains a bacteriostat sold under the tradename IRGACARE MP* (Ciba Specialty Chemicals Corp., Tarrytown, N.Y.), the most pure form of triclosan, a proven, broad spectrum antibacterial drug used effectively in consumer products for more than 30 years. The VICRYL PLUS suture is indicated for use in general soft tissue approximation and/or ligation, except for ophthalmic, cardiovascular and neurological tissues.

Although the efficacy of rhGDF-5-coated sutures has been demonstrated in animal models, it has been appreciated that as rhGDF-5 is freely soluble in aqueous solutions at a pH of less than 4.5, the high solubility of rhGDF-5 in such solutions may limit the coating efficiency of the suture. In particular, there is a concern that rhGDF-5 may be released from the suture in vivo far more rapidly than is desired.

In order to overcome this problem, it has been suggested that coating include a biodegradable carrier such as gelatin along with GDF-5. The carrier in this case would be able to hold the rhGDF-5 on the suture and release it slowly into the wound.

Although the GDF-5/gelatin coated suture effected successful tissue repair, the presence of the gelatin raised a number of concerns: 1) it is a natural animal product and so is not amenable to large scale manufacture; 2) it swells and so takes a long time to dry; 3) it requires heating to about 45° C. to obtain a uniform solution for coating (but the GDF-5 protein may not be stable at that temperature); and 4) the wet coated sutures must be heated or air-dried for longer times to remove the excess moisture, which may affect protein stability and its ease of use in the operating room. Because of these concerns, gelatin has not been regarded as the ideal carrier for coating rhGDF-5 onto sutures.

Therefore, it is a further object of the present invention to select a biodegradable synthetic carrier for coating sutures with rhGDF-5.

The literature reports using sucrose acetate isobutyrate (SAIB) as a controlled release carrier for drug delivery. For example, WO2005100399 (Bing) discloses using SAIB for the sustained release of antibodies. WO2005115438 (Robyn) discloses using SAIB for the sustained release of morphogenic proteins. WO2005107765 (Igo) discloses using SAIB for controlled release of drugs into the pericardial space. WO2003030923 (Van Vlassalaer) discloses using SAIB in a controlled release system. WO2003000282 (Genentech) discloses using SAIB in a controlled system for proteins. WO2004037265 (Allan) discloses using SAIB in a controlled release system; WO20010786683 (Genentech) discloses using SAIB in a controlled release system for growth hormones. U.S. Pat. No. 6,992,065 (Durect) discloses using SAIB in a controlled release system for growth hormones. U.S. Pat. No. 6,911,411 (Akzo Nobel NV) discloses using SAIB in a device for the controlled release of Cefquinome. U.S. Pat. No. 5,747,058 (Southern Biosystems) discusses using SAIB in a high viscosity liquid controlled delivery system. WO 2004052336 A3 (Durect) discusses use of high viscosity liquid controlled delivery system and medical or surgical device. U.S. Pat. No. 6,051,558 (Southern Biosystems) discloses using SAIB in a device for the controlled release of GnRH hormone. US Published Patent Application US20060121113 (Gruenenthal) discloses using SAIB in a controlled release device. EP1274459 B1 (Durect) discloses using SAIB for the controlled release of growth hormones. None of these publications discloses using SAIB as a carrier in a coating upon a suture.

### SUMMARY OF THE INVENTION

The present invention relates to novel sutures having a coating containing i) an active substance and ii) a non-polymeric compound that forms a liquid (preferably, high viscosity) material suitable for the coating and delivery of the biologically active substance in a controlled fashion. The coating materials can optionally be diluted with an ampiphilic solvent to form a material of lower viscosity, making it easier for the material to evenly coat the suture. This solvent is both lipid soluble and water soluble, and rapidly volatilizes to leave behind a thin coating upon the suture.

The coating is generally applied to the suture in liquid form, and contains at least one non-water soluble liquid carrier material, preferably comprising a non-polymeric ester or mixed ester of one or more carboxylic acids, and more preferably having a viscosity of at least 5,000 cP at 37° C. that preferably does not crystallize neat under ambient or physiological conditions. The coating composition can be dissolved in a physiologically acceptable ampiphilic solvent to lower its viscosity, making it easier for the material to coat the suture. After coating of the composition containing solvents upon the suture, the ampiphilic solvent rapidly volatilizes away from the material during drying, and so the coating material thus increases significantly in viscosity, and thereby forms a controlled release matrix for the bioactive substance contained in the coating.

In another aspect, the invention relates to a method of administering a biologically active substance to a human by administering a suture having a composition containing i) a non-water soluble, liquid carrier material comprising a non-polymeric ester or mixed ester of one or more carboxylic acids, preferably having a viscosity of at least 5,000 cP at 37° C., that preferably does not crystallize neat under ambient or physiological conditions and ii) a biologically active substance.

In another aspect, the invention relates to a suture having a coating containing a non-water soluble, liquid carrier material comprising a non-polymeric ester or mixed ester of one or more carboxylic acids, preferably having a viscosity of at least 5,000 cP at 37° C., that preferably does not crystallize neat under ambient or physiological conditions.

In one preferred embodiment of the present invention, there is provided a surgical suture having a coating thereon comprising an effective amount of i) a growth factor (preferably, a bone morphogenetic protein (BMP) such as rhGDF-5) and ii) a biodegradable, non-polymeric, non-water soluble, liquid carrier material, preferably having a viscosity of at least 5,000 cP at 37° C. Such preferred carrier materials are characterized by their ability to dry quickly after they are coated upon the suture under ambient conditions.

The advantage of using such a quick drying, small molecule carrier is that it provides a coating containing stabilized rhGDF-5 without having to expose the proteinaceous rhGDF-5 to heat. This brings the advantages of ease of application and greater protein stability as compared to the previous gelatin-based approach.

In some embodiments, the suture is coated with a composition comprising:
a) a growth factor (preferably, rhGDF-5),
b) a biodegradable, non-polymeric, non-water soluble, liquid carrier material (such as sucrose acetate isobutyrate (SAIB)),
c) a growth factor stabilizer and buffer (such as trehalose and glycine),
d) a solvent in which both the biodegradable, non-polymeric, non-water soluble, liquid carrier material and the protein stabilizer are miscible (preferably, an ampiphilic solvent such as N-methyl pyrrolidone (NMP)),
e) a volatile alcohol (such as ethanol).

One problem confronted by the present inventors was the need to identify a solvent that could solubilize both a hydrophobic carrier material (such as SAIB) and a hydrophilic stabilizer (such as trehalose). The present inventors found that use of an ampiphilic solvent (such as NMP) provided satisfactory solubility for each material.

Therefore, in some embodiments, there is provided a suture that is coated with a composition comprising:
a) a biodegradable, non-polymeric, non-water soluble, liquid carrier material (such as SAIB),
b) a growth factor stabilizer and buffer (such as trehalose and glycine),
c) an ampiphilic solvent (such as NMP),

However, another problem confronting the present inventors was that use of the ampiphilic solvent NMP by itself led to somewhat long drying times (on the order of 8-10 minutes). Typically, a drying time on the order of less than about 5 minutes is desired, and less than 2 minutes is even more desired. The present inventors found that adding a volatile alcohol (such as ethanol) reduced the drying time of the composition to around 10 seconds.

Therefore, in some embodiments, there is provided a suture coated with a composition comprising:
a) an ampiphilic solvent (such as NMP), and
b) a volatile alcohol (such as ethanol).

### DETAILED DESCRIPTION OF THE INVENTION

For the purposes of the present invention, “non-polymeric” is considered to be less than about 2000 daltons. The terms “non-polymeric” and small molecule” are used interchangeably.

The biodegradable, non-polymeric, non-water soluble, liquid carrier materials of the present invention include, but are not limited to sucrose acetate isobutyrate (SAIB), sucrose acetate, sucrose octa acetate, dioctyladipate, medium and long chain fatty acid esters with 10-24 carbon atoms, medium and long chain phospholipids with 10-24 carbon atoms, medium and long chain diglycerides with 10-24 carbon atoms, medium and long chain triglycerides with 10-24 carbon atoms, butyl phthalate esters, sterol esters, steroid esters and vitamin E esters.

Many of the biodegradable, non-polymeric, non-water soluble, liquid carrier materials described directly above are high viscosity materials. One exception is that of the phospholipids class. These materials are generally very expensive. Therefore, in many preferred embodiments, the biodegradable, non-polymeric, non-water soluble, liquid carrier material is a high viscosity liquid carrier material (HVLCM)

### Non-Water-Soluble, High Viscosity, Liquid Carriers

In a preferred embodiment, the high viscosity liquid carrier material (HVLCM) is non-polymeric, non-water soluble, and has a viscosity of at least 5,000 cP, (and optionally at least 10,000, 15,000; 20,000; 25,000 or even 50,000 cP) at 37° C. that does not crystallize neat under ambient or physiological conditions. The term “non-water soluble” refers to a material that is soluble in water to a degree of less than one percent by weight under ambient conditions. The term “non-polymeric” refers to esters or mixed esters having essentially no repeating units in the acid moiety of the ester, as well as esters or mixed esters having acid moieties wherein functional units in the acid moiety are repeated a small number of times (i.e., oligomers). Generally, materials having more than five identical and adjacent repeating units (or mers) in the acid moiety of the ester are excluded by the term “non-polymeric” as used herein, but materials containing dimers, trimers, tetramers, or pentamers are included within the scope of this term. When the ester is formed from hydroxy-containing carboxylic acid moieties that can further esterify, such as lactic acid or glycolic acid, the number of repeat units is calculated based upon the number of lactide or glycolide moieties, rather than upon the number of lactic acid or glycolic acid moieties, where a lactide repeat unit contains two lactic acid moieties esterified by their respective hydroxy and carboxy moieties, and where a glycolide repeat unit contains two glycolic acid moieties esterified by their respective hydroxy and carboxy moieties. Esters having 1 to about 20 etherified polyols in the alcohol moiety thereof, or 1 to about 10 glycerol moieties in the alcohol moiety thereof, are considered non-polymeric as that term is used herein.

In a particular embodiment, the high viscosity liquid carrier material (HVLCM) decreases in viscosity, in some cases significantly, when mixed with a solvent to form a low viscosity liquid carrier material (LVLCM) that can be mixed with a biologically active substance and used as a coating upon a suture for controlled delivery of the active substance. The LVLCM/biologically active substance composition is typically easier to use as a coating than a HVLCM/biologically active substance composition, because it flows more easily onto and around the suture. The LVLCM can have any desired viscosity. It is believed that a viscosity range for the LVLCM of less than approximately 6,000 cP, more particularly, less than approximately 4,000 cP, even more particularly, less than approximately 1,000 cP, and yet even more particularly less than 200 cP, is typically useful.

A preferred HVLCM used in the present invention can be one or more of a variety of materials. Suitable materials include non-polymeric esters or mixed esters of one or more carboxylic acids. In a particular embodiment, the ester is formed from carboxylic acids that are esterified with a polyol having from about 2 to about 20 hydroxy moieties, and which may include 1 to about 20 etherified polyols. Particularly suitable carboxylic acids for forming the acid moiety of the ester of the HVLCM include carboxylic acids having one or more hydroxy groups, e.g., those obtained by ring opening alcoholysis of lactones, or cyclic carbonates or by the alcoholysis of carboxylic acid anhydrides. Amino acids are also suitable for forming esters with the polyol. In a particular embodiment, the ester or mixed ester contains an alcohol moiety having one or more terminal hydroxy moieties that have been esterified with one or more carboxylic acids obtained by alcoholysis of a carboxylic acid anhydride, such as a cyclic anhydride.

Nonlimiting examples of suitable carboxylic acids that can be esterified to form the HVLCM of the invention include glycolic acid, lactic acid, ε-hydroxycaproic acid, serine, and any corresponding lactones or lactams, trimethylene carbonate, and dioxanone. The hydroxy-containing acids may themselves be further esterified through the reaction of their hydroxy moieties with additional carboxylic acid moieties, which may be the same as or different from other carboxylic acid moieties in the material. Suitable lactones include, but are not limited to, glycolide, lactide, ε-caprolactone, butyrolactone, and valerolactone. Suitable carbonates include but are not limited to trimethylene carbonate and propylene carbonate.

The alcohol moiety of the ester or mixed ester may be derived from a polyhydroxy alcohol having from about 2 to about 20 hydroxy groups, and as indicated above, may be formed by etherifying 1 to 20 polyol molecules. Suitable alcohol moieties include those derived by removing one or more hydrogen atoms from: monofunctional C₁-C₂₀ alcohols, difunctional C₁-C₂₀ alcohols, trifunctional alcohols, hydroxy-containing carboxylic acids, hydroxy-containing amino acids, phosphate-containing alcohols, tetrafunctional alcohols, sugar alcohols, monosaccharides, disaccharides, sugar acids, and polyether polyols. More specifically, the alcohol moieties may include one or more of: dodecanol, hexanediol, more particularly, 1,6-hexanediol, glycerol, glycolic acid, lactic acid, hydroxybutyric acid, hydroxyvaleric acid, hydroxycaproic acid, serine, ATP, pentaerythritol, mannitol, sorbitol, glucose, fructose, sucrose, glucuronic acid, polyglycerol ethers containing from 1 to about 10 glycerol units, polyethylene glycols containing 1 to about 20 ethylene glycol units.

In particular embodiments of the invention, at least one of the carboxylic acid moieties of the esters or mixed esters of the invention comprise at least one oxy moiety. In an even more particular embodiment, each of the carboxylic acid moieties comprise at least one oxy moiety.

In another particular embodiment, at least one of the carboxylic acid moieties of the esters or mixed esters of the invention contains 2 to 4 carbon atoms. In an even more particular embodiment, each of the carboxylic acid moieties of the esters or mixed esters of the invention contains 2 to 4 carbon atoms.

In another more particular embodiment of the invention, at least one of the carboxylic acid moieties of the ester or mixed ester of the invention has 2 to 4 carbon atoms and contains at least one oxy moiety. In another more particular embodiment of the invention, each of the carboxylic acid moieties of the ester or mixed ester of the invention has 2 to 4 carbon atoms and contains at least one oxy moiety.

The acyl groups forming the acyloxy substituents of the invention may be any moiety derived from a carboxylic acid in accordance with the commonly accepted definition of the term “acyl.”

The use of relatively small chain (2 to 6 carbon atoms), oxy-substituted carboxylic acid moieties in the ester or mixed ester of the invention is advantageous. When these acid moieties are present in the form of oligomeric esters (i.e., a subsequent acid moiety joined to the previous acid moiety through esterification of the subsequent carboxy with the previous oxy), hydrolysis of the material is considerably easier than for oligomers made with more than 6 carbon atoms because the material is more hydrophilic. In general, for drug delivery it is desired that the HVLCM be water insoluble, but somewhat hydrophilic. In general, HVLCMs synthesized with more hydrophilic units (as determined by a higher O:C ratio) will be expected to absorb water more rapidly and degrade more quickly. For example, a HVLCM made by covalently linking 4 moles of glycolide to one mole of glycerol will be expected to absorb water more rapidly and degrade more quickly than a HVLCM made by covalently linking 2 moles of glycolide and 2 moles of lactide to one mole of glycerol. Similar increases can be expected for more flexible molecules and for more branched, spherical molecules based on free volume arguments. Use of flexible and branched molecules may also have the benefit of lowering the viscosity of the LVLCM. Using carboxylic acids and/or polyols of different chain length and using carboxylic acids having oxy-substitution allows a precise control of the degree of hydrophilicity and of the solubility of the resulting ester. These materials are sufficiently resistant to dissolution in vivo that they are able to provide a controlled release of bioactive substances into the body accompanied or followed by oxy bonds hydrolyzing in vivo.

In an even more particular embodiment, the invention excludes the acetate and isobutyrate ester of sucrose having a ratio of acetate to isobutyrate acid moieties of 2:6. However, sucrose acetate isobutyrate ester having a ratio of acetate to isobutyrate moieties of 2:6 is included within the scope of the invention for use in aerosol formulations, as well as for the delivery of lysozyme, paclitaxel, 5-fluorouracil, and antiretroviral drugs like AZT and ddC. This material can be made according to the procedures described in U.S. Pat. No. 2,931,802.

In general, the HVLCM esters of the invention can be made by reacting one or more alcohols, in particular one or more polyols, which will form the alcohol moiety of the resulting esters with one or more carboxylic acids, lactones, lactams, carbonates, or anhydrides of the carboxylic acids which will form the acid moieties of the resulting esters. The esterification reaction can be conducted simply by heating, although in some instances addition of a strong acid or strong base esterification catalyst may be used. Alternatively, an esterification catalyst such as stannous 2-ethylhexanoate can be used. The heated reaction mixture, with or without catalyst, is heated with stirring, then dried, e.g., under vacuum, to remove any unreacted starting materials, to produce a liquid product. Sucrose acetate isobutyrates can be made by following the procedures described in U.S. Pat. No. 2,931,802.

In this regard, the polyol can be viewed as an oligomerization initiator, in the sense that it provides a substrate for esterification of carboxylic acids, in particular, of oligomers of lactide, glycolide, or other esterified hydroxy-substituted carboxylic acids.

In some preferred embodiments, the carrier is SAIB. SAIB is desirable because it forms a thin coat on the suture, and remains on the suture during normal handling. SAIB is also desirable because it has been found that a preferred growth factor (GDF-5) is soluble in SAIB and thereby is able to uniformly disperse on the suture. Lastly, SAIB is desirable because it produces a relatively flexible coat on the suture.

The HVLCM (and preferably the SAIB) is typically added to the compositions in an amount in the range from about 1 percent to about 95 percent by weight, more particularly from about 5 to about 90 wt %, relative to the total weight of the composition. Even more particularly, the solvent is present in the composition in an amount in the range from about 10 percent to about 90 percent by weight. Other particular ranges include from about 30 percent to 70 percent by weight, and from about 40 to about 60 percent by weight. In one especially preferred embodiment, the SAIB is present at a concentration of about 50 weight percent.

Preliminary experiments found that the solubility time of the composition slightly increased as the concentration of SAIB was increased from 10 wt % to 25 wt % to about 50 wt %, while the drying time was not really affected (it was still less than one minute). Thus, compositions within this range of SAIB are preferred. Because the 50 wt % SAIB composition produced a very satisfactory coat upon the suture, it was selected for further study.

### Solvents

As described above, in one embodiment of the invention, the HVLCM can be mixed with a viscosity lowering solvent to form a lower viscosity liquid carrier material (LVLCM), which can then be mixed with the biologically active substance to form a composition for coating the suture. The solvent should provide three qualities. First, each of the other components of the composition should be soluble in the solvent. This quality provides for a uniform coating. Second, each of the other components of the composition should solubilize in the solvent within about 5 minutes (“solubility time”). Lastly, once the composition is applied to the suture, the solvent should evaporate off the suture within about 5 minutes (“drying time”). Low solubility and drying times allow the surgeon to intraoperatively coat a suture of choice within an acceptable time frame.

In some preferred embodiments, the solvent is able to solubilize both a hydrophobic carrier material (such as SAIB) and a hydrophilic stabilizer (such as trehalose/glycine). The present inventors found that use of an ampiphilic solvent (such as NMP) provided satisfactory solubility for each material. Moreover, the solubility time of such systems was also found to be acceptable (i.e., within about 2-3 minutes). Therefore, in some embodiments, the solvent is an ampiphilic solvent . Suitable ampiphilic solvents include N-methyl-2-pyrrolidone (NMP), dimethyl sulfoxide (DMSO), and N-N-dimethyl formamide (DMF). N-methyl-2-pyrrolidone is particularly preferred because it is non-toxic and is already present in an FDA approved formulation.

Generally, the solvent can be water soluble, non-water soluble, or water miscible, and can include, acetone, benzyl alcohol, benzyl benzoate, N-(betahydromethyl)lactamide, butylene glycol, caprolactam, caprolactone, corn oil, decylmethylsulfoxide, dimethyl ether, dimethyl sulfoxide, 1-dodecylazacycloheptan-2-one, ethanol, ethyl acetate, ethyl lactate, ethyl oleate, glycerol, glycofurol(tetraglycol), isopropyl myristate, methyl acetate, methyl ethyl ketone, N-methyl-2-pyrrolidone, MIGLYOLs (esters of caprylic and/or capric acids with glycerol or alkylene glycols, e.g., MIGLYOL 810 or 812 (caprylic/capric triglycerides), MIGLYOL 818 (caprylic/capric/linoleic triglyceride), MIGLYOL 829 (caprylic/capric/succinic triglyceride), MIGLYOL 840 (propylene glycol dicaprylate/caprate)), oleic acid, peanut oil, polyethylene glycol, propylene carbonate, 2-pyrrolidone, sesame oil, SOLKETAL (-2,2-dimethyl-1,3-dioxolane-4-methanol), tetrahydrofuran, TRANSCUTOL (diethylene glycol monoethyl ether, carbitol), triacetin, triethyl citrate, and combinations thereof. Particularly suitable solvents and/or propellants include benzyl benzoate, dimethyl sulfoxide, ethanol, ethyl lactate, glycerol, glycofurol(tetraglycol), N-methyl-2-pyrrolidone, MIGLYOL 810, polyethylene glycol, propylene carbonate, 2-pyrrolidone, and tetrafluoroethane.

Additionally, if the composition is to be applied to the suture as an aerosol, the solvent may be or may include one or more propellants, such as CFC propellants like trichlorofluoromethane and dichlorofluoromethane, non-CFC propellants like tetrafluoroethane (R-134a), 1,1,1,2,3,3,3-heptafluoropropane (R-227), dimethyl ether, propane, and butane.

When the coating composition is used as a LVLCM in conjunction with a biologically active substance, it should contain a solvent that the HVLCM is soluble in. In certain instances, the active substance to be delivered is also soluble in the solvent. The solvent should be non-toxic and otherwise biocompatible.

When esters of 1,6-hexanediol or glycerol are used as the HVLCM, some possible solvents are ethanol, N-methylpyrrolidone, propylene carbonate, and PEG 400.

The solvent is typically added to the compositions in an amount in the range from about 1 percent to about 95 percent by weight, more particularly from about 5 to about 90 wt %, relative to the total weight of the composition. Even more particularly, the solvent is present in the composition in an amount in the range from about 10 percent to about 55 percent by weight. Other particular ranges include from about 10 percent to 50 percent by weight, and from about 10 to about 30 percent by weight.

Although dissolution in ampiphilic solvent is particularly useful with non-polymeric esters or mixed esters having very high viscosities, e.g., on the order of 100,000 cP at 37° C., some nonpolymeric esters or mixed esters suitable for use in the invention, while having viscosities above 5,000 cP at 37° C., are not as viscous, and may be applied as a coating neat, i.e., without the addition of a solvent.

### Alcohol

However, another problem confronting the present inventors was the low drying time of the ampiphilic solvent NMP. Because NMP is relatively non-volatile, its use by itself led to somewhat long drying times (on the order of 8-10 minutes). Typically, a drying time on the order of less than about 5 minutes is desired. The present inventors found that adding a volatile alcohol (such as ethanol) to the composition increased the volatility of the NMP and thereby reduced the drying time of the composition to around 10 seconds.

Therefore, in some embodiments, the composition of the present invention includes a volatile alcohol. Exemplary alcohols include ethanol, isopropanol, and n-propanol. The alcohol is typically added to the compositions in an amount in the range from about 1 percent to about 10 percent by weight, more particularly from about 3 to about 7 wt %, relative to the total weight of the composition. Even more particularly, the alcohol is present in the composition in an amount in the range from about 4 percent to about 6 percent by weight.

### Active Substance

When the HVLCM or LVLCM is to be used as a vehicle for delivery or controlled release of an active substance, this substance may be any substance that exhibits a desired property. In a particular embodiment, the substance is a biologically active substance.

The term “biologically active substance” as used herein refers to an inorganic or organic molecule including a drug, peptide, protein, carbohydrate (including monosaccharides, oligosaccharides, and polysaccharides), nucleoprotein, mucoprotein, lipoprotein, synthetic polypeptide or protein, or a small molecule linked to a protein, glycoprotein, steroid, nucleic acid (any form of DNA, including cDNA, or RNA, or a fragment thereof), nucleotide, nucleoside, oligonucleotides (including antisense oligonucleotides), gene, lipid, hormone, vitamin, including vitamin C and vitamin E, or combination thereof, that causes a biological effect when administered in vivo to an animal, including but not limited to birds and mammals, including humans.

Suitable proteins include, but are not limited to, human growth hormone, fibroblast growth factor (FGF), erythropoietin (EPO), platelet derived growth factor (PDGF), granulocyte colony stimulating factor (g-CSF), bovine somatotropin (BST), tumor necrosis factor (TNF), members of the transforming growth factor-beta (TGF-β) superfamily, interleukins, insulin, and interferon.

### Growth Factors

In some embodiments, the “biologically active substance” is a growth factor. As used herein, the term “growth factor” encompasses any cellular product that modulates the growth or differentiation of other cells, particularly connective tissue progenitor cells. The growth factors that may be used in accordance with the present invention include, but are not limited to, members of the fibroblast growth factor family, including acidic and basic fibroblast growth factor (FGF-1 and -2) and FGF-4, members of the platelet-derived growth factor (PDGF) family, including PDGF-AB, PDGF-BB and PDGF-AA; EGFs, members of the insulin-like growth factor (IGF) family, including IGF-I and -II;, the TGF-β superfamily, including TGF-β1, 2 and 3 (including MP-52), osteoid-inducing factor (OIF), angiogenin(s), endothelins, hepatocyte growth factor and keratinocyte growth factor; members of the bone morphogenetic proteins (BMPs) BMP-1; BMP-3; BMP-2; OP-1; BMP-2A, -2B, -4, -7 and -14; GDF-5; HBGF-1 and HBGF-2; growth differentiation factors (GDFs), members of the hedgehog family of proteins, including indian, sonic and desert hedgehog; ADMP-1; and members of the colony-stimulating factor (CSF) family, including CSF-1, G-CSF, and GM-CSF; and isoforms thereof.

In some embodiments, the growth factor is a BMP. BMPs disclosed in U.S. Pat. No. 6,936,582, the specification of which is incorporated by reference in its entirety, are contemplated for use in the present invention.

The OP/BMP morphogens of the present invention are naturally occurring proteins, or functional variants of naturally occurring proteins, in the osteogenic protein/bone morphogenetic protein (OP/BMP) family within the TGF-β superfamily of proteins. That is, these proteins form a distinct subgroup, referred to herein as the “OP/BMP morphogens,” within the loose evolutionary grouping of sequence-related proteins known as the TGF-β superfamily. Members of this protein family comprise secreted polypeptides that share common structural features, and that are similarly processed from a pro-protein to yield a carboxy-terminal mature protein. Within the mature protein, all members share a conserved pattern of six or seven cysteine residues defining a 97-106 amino acid domain, and the active form of these proteins is either a disulfide-bonded homodimer of a single family member, or a heterodimer of two different members. See, e.g., Massague, Annu. Rev. Cell Biol. 6:597 (1990); Sampath et al., J. Biol. Chem. 265:13198 (1990). For example, in its mature, native form, natural-sourced human OP-1 is a glycosylated dimer typically having an apparent molecular weight of about 30-36 kDa as determined by SDS-PAGE. When reduced, the 30 kDa protein gives rise to two glycosylated peptide subunits having apparent molecular weights of about 16 kDa and 18 kDa. The unglycosylated protein has an apparent molecular weight of about 27 kDa. When reduced, the 27 kDa protein gives rise to two unglycosylated polypeptide chains, having molecular weights of about 14 kDa to 16 kDa.

Typically, the naturally occurring OP/BMP proteins are translated as a precursor, having an N-terminal signal peptide sequence, a “pro” domain, and a “mature” protein domain. The signal peptide is typically less than 30 residues, and is cleaved rapidly upon translation at a cleavage site that can be predicted using the method of Von Heijne, Nucleic Acids Research 14:4683-4691 (1986). The “pro” domain is variable both in sequence and in length, ranging from approximately 200 to over 400 residues. The pro domain is cleaved to yield the “mature” C-terminal domain of approximately 115-180 residues, which includes the conserved six- or seven-cysteine C-terminal domain of 97-106 residues. As used herein, the “pro form” of an OP/BMP family member includes a protein comprising a folded pair of polypeptides, each comprising a pro domain in either covalent or noncovalent association with the mature domains of the OP/BNP polypeptide. Typically, the pro form of the protein is more soluble than the mature form under physiological conditions. The pro form appears to be the primary form secreted from cultured mammalian cells. The “mature form” of the protein includes a mature C-terminal domain which is not associated, either covalently or noncovalently, with the pro domain. Any preparation of OP-1 is considered to contain mature form when the amount of pro domain in the preparation is no more than 5% of the amount of “mature” C-terminal domain.

OP/BMP family members useful herein include any of the known naturally-occurring native proteins including allelic, phylogenetic counterparts and other variants thereof, whether naturally-sourced or biosynthetically produced (e.g., including “muteins” or “mutant proteins”), as well as new, active members of the OP/BMP family of proteins.

Particularly useful sequences include those comprising the C-terminal seven cysteine domains of mammalian, preferably human, human OP-1, OP-2, OP-3, BMP2, BMP3, BMP4, BMP5, BMP6, BMP8 and BMP9. Other proteins useful in the practice of the invention include active forms of GDF-5, GDF-6, GDF-7, DPP, Vg1, Vgr-1, 60A, GDF-1, GDF-3, GDF-5, GDF-6, GDF-7, BMP10, BMP11, BMP13, BMP15, UNIVIN, NODAL, SCREW, ADMP or NURAL and amino acid sequence variants thereof. In one currently preferred embodiment, the OP/BMP morphogens of the invention are selected from any one of: OP-1, OP-2, OP-3, BMP2, BMP3, BMP4, BMP5, BMP6, and BMP9.

Publications disclosing these sequences, as well as their chemical and physical properties, include: OP-1 and OP-2: U.S. Pat. No. 5,011,691, U.S. Pat. No. 5,266,683, and Ozkaynak el al., EMBO J. 9:2085-2093 (1990); OP-3: WO94/10203; BMP2, BMP3, and BMP4: U.S. Pat. No. 5,013,649, WO91/18098, WO88/00205, and Wozney et al., Science 242:1528-1534 (1988); BMP5 and BMP6: WO90/11366 and Celeste et aL, Proc. Natl. Acad. Sci. (USA) 87:9843-9847 (1991); Vgr-1: Lyons et at., Proc. Natl. Acad. Sci. (USA) 86:4554-4558 (1989); DPP: Padgett et al., Nature 325:81-84 (1987); Vg1: Weeks, Cell 51:861-867 (1987); BMP9: WO95/33830; BMP10: WO94/26893; BMP-11: WO94/26892; BMP12: WO95/16035; BMP-13 WO95/16035, GDF-1: WO92/00382 and Lee et al., Proc. Natl. Acad. Sci (USA) 88:4250-4254 (1991); GDF-8: WO94/21681; GDF-9: WO94/15966; GDF-10: WO95/10539; GDF-11: WO96/01845; BMP-15: WO96/36710; MP121: WO96/01316; GDF-5 (CDMP-1, MP52): WO94/15949, WO96/14335, WO93/16099 and Storm el al., Nature 368:639-643 (1994); GDF-6 (CDMP-2, BMP13): WO95/01801, WO96/14335 and WO95/10635; GDF-7 (CDMP-3, BMP12): WO95/10802 and WO95/10635; BMP-3b: Takao et al., Biochem. Biophys. Res. Comm. 219:656-662 (1996); GDF-3: WO94/15965; 60A: Basler et al., Cell 73:687-702 (1993) and GenBank Accession No. L12032. In another embodiment, useful proteins include biologically active biosynthetic constructs, including novel biosynthetic proteins and chimeric proteins designed using sequences from two or more known OP/BNT family proteins. See also the biosynthetic constructs disclosed in U.S. Pat. No. 5,011,691, the disclosure of which is incorporated herein by reference (e.g., COP-1, COP-3, COP-4, COP-5, COP-7, and COP-16).

In other preferred embodiments, the OP/BMP morphogens useful herein include proteins which comprise an amino acid sequence sharing at least 70% amino acid sequence “homology” and, preferably, 75% or 80% homology with the C-terminal seven cysteine domain present in the active forms of human OP-1 (i.e., residues 330-431, as shown in SEQ ID NO: 2 of U.S. Pat. No. 5,266,683) or GDF-5. In other preferred embodiments, the OP/BMP morphogens useful herein include proteins which comprise an amino acid sequence sharing at least 60% amino acid sequence identity and, preferably, 65% or 70% identity with the C-terminal seven cysteine domain present in the active forms of human OP-1 or GDF-5. Thus, a candidate amino acid sequence can be aligned with the amino acid sequence of the C-terminal seven cysteine domain of human OP-1 using the method of Needleman el al., J. Mol. Biol. 48:443-453 (1970), implemented conveniently by computer programs such as the Align program (DNAstar, Inc.). As will be understood by those skilled in the art, homologous or functionally equivalent sequences include functionally equivalent arrangements of the cysteine residues within the conserved cysteine skeleton, including amino acid insertions or deletions which alter the linear arrangement of these cysteines, but do not materially impair their relationship in the folded structure of the dimeric protein, including their ability to form such intra- or inter-chain disulfide bonds as may be necessary for biological activity. Therefore, internal gaps and amino acid insertions in the candidate sequence are ignored for purposes of calculating the level of amino acid sequence homology or identity between the candidate and reference sequences.

“Amino acid sequence homology” is understood herein to include both amino acid sequence identity and similarity. Thus, as used herein, a percentage “homology” between two amino acid sequences indicates the percentage of amino acid residues, which are identical or similar between the sequences. “Similar” residues are “conservative substitutions” which fulfill the criteria defined for an “accepted point mutation” in Dayhoffel al., Atlas of Protein Sequence and Structure Vol. 5 (Suppl. 3), pp. 354-352 (1978), Natl. Biomed. Res. Found., Washington, D.C. Thus, “conservative amino acid substitutions” are residues that are physically or functionally similar to the corresponding reference residues, having similar size, shape, electric charge, and/or chemical properties such as the ability to form covalent or hydrogen bonds, or the like. Examples of conservative substitutions include the substitution of one amino acid for another with similar characteristics, e.g., substitutions within the following groups. (a) valine, glycine, (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine. The term “conservative substitution” or “conservative variation” also includes the use of a substituted amino acid in place of an unsubstituted parent amino acid in a given polypeptide chain, provided that the resulting substituted polypeptide chain has biological activity useful in the present invention.

The OP/BMP morphogens of the invention are characterized by biological activities which may be readily ascertained by those of ordinary skill in the art.

The OP/BMP morphogens contemplated herein can be expressed from intact or truncated genomic or cDNA or from synthetic DNAs in prokaryotic or eukaryotic host cells. The dimeric proteins can be isolated from the culture media and/or refolded and dimerized in vitro to form biologically active preparations. Heterodimers can be formed in vitro by combining separate, distinct polypeptide chains. Alternatively, heterodimers can be formed in a single cell by co-expressing nucleic acids encoding separate, distinct polypeptide chains. See, for example, WO93/09229, or U.S. Pat. No. 5,411,941, for several exemplary recombinant heterodimer protein production protocols. Currently preferred host cells include, without limitation, prokaryotes including E. coli, or eukaryotes including yeast such as Saccharomyces, insect.cells, or mammalian cells, such as CHO, COS or BSC cells. One of ordinary skill in the art will appreciate that other host cells can be used to advantage. Detailed descriptions of the proteins useful in the practice of this invention, including how to make, use and test them for activity, are disclosed in numerous publications, including U.S. Pat. Nos. 5,266,683 and 5,011,691, the disclosures of which are herein incorporated by reference.

In some embodiments, the growth factor is GDF-5. When GDF-5 is selected as the growth factor, it may be combined with a PLGA carrier.

The term drug, as used herein, refers to any substance used internally as a medicine for the treatment, cure, or prevention of a disease or disorder, and includes but is not limited to immunosuppressants, antioxidants, anesthetics, analgesics, chemotherapeutic agents, steroids (including retinoids), hormones, antibiotics, antivirals, antifungals, antiproliferatives, antihistamines, anticoagulants, antiphotoaging agents, melanotropic peptides, nonsteroidal and steroidal anti-inflammatory compounds, antipsychotics, and radiation absorbers, including UV-absorbers.

Non-limiting examples of pharmacological materials include anti-infectives such as nitrofurazone, sodium propionate, antibiotics, including penicillin, tetracycline, oxytetracycline, chlorotetracycline, bacitracin, nystatin, streptomycin, neomycin, polymyxin, gramicidin, chloramphenicol, erythromycin, and azithromycin; sulfonamides, including sulfacetamide, sulfamethizole, sulfamethazine, sulfadiazine, sulfamerazine, and sulfisoxazole, and anti-virals including idoxuridine; antiallergenics such as antazoline, methapyritene, chlorpheniramine, pyrilamine prophenpyridamine, hydrocortisone, cortisone, hydrocortisone acetate, dexamethasone, dexamethasone 21-phosphate, fluocinolone, triamcinolone, medrysone, prednisolone, prednisolone 21-sodium succinate, and prednisolone acetate; desensitizing agents such as ragweed pollen antigens, hay fever pollen antigens, dust antigen and milk antigen; vaccines such as smallpox, yellow fever, distemper, hog cholera, chicken pox, antivenom, scarlet fever, diphtheria toxoid, tetanus toxoid, pigeon pox, whooping cough, influenzae rabies, mumps, measles, poliomyelitic, and Newcastle disease; decongestants such as phenylephrine, naphazoline, and tetrahydrazoline; miotics and anticholinesterases such as pilocarpine, esperine salicylate, carbachol, diisopropyl fluorophosphate, phospholine iodide, and demecarium bromide; parasympatholytics such as atropine sulfate, cyclopentolate, homatropine, scopolamine, tropicamide, eucatropine, and hydroxyamphetamine; sympathomimetics such as epinephrine; sedatives and hypnotics such as pentobarbital sodium, phenobarbital, secobarbital sodium, codeine, (a-bromoisovaleryl)urea, carbromal; psychic energizers such as 3-(2-aminopropyl) indole acetate and 3-(2-aminobutyl) indole acetate; tranquilizers such as reserpine, chlorpromayline, and thiopropazate; anesthetics, such as novicaine and bupivacaine; androgenic steroids such as methyl-testosterone and fluorymesterone; estrogens such as estrone, 17-flestradiol, ethinyl estradiol, and diethyl stilbestrol; progestational agents such as progesterone, megestrol, melengestrol, chlormadinone, ethisterone, norethynodrel, 19-norprogesterone, norethindrone, medroxyprogesterone and 17-0-hydroxy-progesterone; humoral agents such as the Prostaglandins, for example PGEI, PGE2 and PGF2; antipyretics such as aspirin, sodium salicylate, and salicylamide; antispasmodics such as atropine, methantheline, papaverine, and methscopolamine bromide; antimalarials such as the 4-aminoquinolines, 8-aminoquinolines, chloroquine, and pyrimethamine, antihistamines such as diphenhydramine, dimenhydrinate, tripelennamine, perphenazine, and chlorphenazine; cardioactive agents such as dibenzhydroflume thiazide, flumethiazide, chlorothiazide, and aminotrate; nutritional agents such as vitamins, natural and synthetic bioactive peptides and proteins, including growth factors, cell adhesion factors, cytokines, and biological response modifiers.

The active compound is included in the composition in an amount sufficient to deliver to the host patient an effective amount to achieve a desired effect. The amount of drug or biologically active agent incorporated into the composition depends upon the desired release profile, the concentration of drug required for a biological effect, and the desired period of release of the drug.

The concentration of active compound in the composition will also depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The composition may be administered in one dosage, or may be divided into a number of smaller doses to be administered at varying intervals of time.

The biologically active substance is typically present in the composition in the range from about 0.1 percent to about 20 percent by weight, more particularly from about 0.5 percent to about 20 percent by weight relative to the total weight of the composition, and more typically, between approximately 1 percent to about 15 percent by weight, and more. Another preferred range is from about 2 percent to about 10 percent by weight. For very active agents, such as growth factors, preferred ranges are less than 1% by weight, and less than 0.0001%.

### Additives

A variety of additives can optionally be added to the HVLCM or LVLCM to modify the properties of the material as desired, and in particular to modify the release properties of the composition with respect to biologically active substances contained therein. The additives can be present in any amount which is sufficient to impart the desired properties to the composition. The amount of additive used will in general be a function of the nature of the additive and the effect to be achieved, and can be easily determined by the skilled artisan. Suitable additives are described in U.S. Pat. No. 5,747,058, the entire contents of which are hereby incorporated by reference. More particularly, suitable additives include water, biodegradable polymers, non-biodegradable polymers, natural oils, synthetic oils, carbohydrates or carbohydrate derivatives, inorganic salts, BSA (bovine serum albumin), surfactants, organic compounds, such as sugars, and organic salts, such as sodium citrate. Some of these classes of additives are described in more detail below. In general, the less water soluble, i.e., the more lipophilic, the additive, the more it will decrease the rate of release of the substrate, compared to the same composition without the additive. In addition, it may be desirable to include additives that increase properties such as the strength or the porosity of the composition.

The addition of additives can also be used to lengthen the delivery time for the active ingredient, making the composition suitable for treatment of disorders or conditions responsive to longer term administration. Suitable additives in this regard include those disclosed in U.S. Pat. No. 5,747,058. In particular, suitable additives for this purpose include polymeric additives, such as cellulosic polymers and biodegradable polymers. Suitable cellulosic polymers include cellulose acetates, cellulose ethers, and cellulose acetate butyrates. Suitable biodegradable polymers include polylactones, polyanhydrides, and polyorthoesters, in particular, polylactic acid, polyglycolic acid, polycaprolactone, and copolymers thereof.

When present, the additive is typically present in the compositions in an amount in the range from about 0.01 percent to about 20 percent by weight, more particularly from about 0.1 percent to about 20 percent by weight, relative to the total weight of the composition, and more typically, is present in the composition in an amount in the range from about 1, 2, or 5 percent to about 10 percent by weight. Certain additives, such as buffers, are only present in small amounts in the composition.

The following categories are nonlimiting examples of classes of additives that can be employed in the composition.

One category of additives are biodegradable polymers and oligomers. The polymers can be used to alter the release profile of the substance to be delivered, to add integrity to the composition, or to otherwise modify the properties of the composition. Non-limiting examples of suitable biodegradable polymers and oligomers include: poly(lactide), poly(lactide-coglycolide), poly(glycolide), poly(caprolactone), polyamides, polyanhydrides, polyamino acids, polyorthoesters, polycyanoacrylates, poly(phosphazines), poly(phosphoesters), polyesteramides, polydioxanones, polyacetals, polyketals, polycarbonates, polyorthocarbonates, degradable polyurethanes, polyhydroxybutyrates, polyhydroxyvalerates, polyalkylene oxalates, polyalkylene succinates, poly(malic acid), chitin, chitosan, and copolymers, terpolymers, oxidized cellulose, or combinations or mixtures of the above materials.

Examples of poly(α-hydroxy acid)s include poly(glycolic acid), poly(DL-lactic acid) and poly(L-lactic acid), and their copolymers. Examples of polylactones include poly(ε-caprolactone), poly(δ-valerolactone) and poly(χ-butyrolactone).

Another additive for use with the present compositions are non-biodegradable polymers. Non-limiting examples of nonerodible polymers which can be used as additives include: polyacrylates, ethylene-vinyl acetate polymers, cellulose and cellulose derivatives, acyl substituted cellulose acetates and derivatives thereof, non-erodible polyurethanes, polystyrenes, polyvinyl chloride, polyvinyl fluoride, polyvinyl(imidazole), chlorosulphonated polyolefins, polyethylene oxide, and polyethylene.

Preferred non-biodegradable polymers include polyvinyl pyrrolidone, ethylene vinylacetate, polyethylene glycol, cellulose acetate butyrate (“CAB”) and cellulose acetate propionate (“CAP”).

A further class of additives which can be used in the present coating compositions are natural and synthetic oils and fats. Oils derived from animals or from plant seeds or nuts typically include glycerides of the fatty acids, chiefly oleic, palmitic, stearic, and linoleic. As a rule, the more hydrogen the molecule contains the thicker the oil becomes.

Non-limiting examples of suitable natural and synthetic oils include vegetable oil, peanut oil, medium chain triglycerides, soybean oil, almond oil, olive oil, sesame oil, peanut oil, fennel oil, camellia oil, corn oil, castor oil, cotton seed oil, and soybean oil, either crude or refined, and medium chain fatty acid triglycerides.

Fats are typically glyceryl esters of higher fatty acids such as stearic and palmitic. Such esters and their mixtures are solids at room temperatures and exhibit crystalline structure. Lard and tallow are examples. In general, oils and fats increase the hydrophobicity of the HVLCM, slowing degradation and water uptake.

Another class of additives which can be used in the present compositions are carbohydrates and carbohydrate derivatives. Non-limiting examples of these compounds include monosaccarides (simple sugars such as fructose and its isomer glucose (dextrose); disaccharides such as sucrose, maltose, cellobiose, and lactose; and polysaccharides.

### Stabilizers and Buffers

When the active substance is a protein such as a growth factor, there is a danger that the protein in solution will be susceptible to denaturation and aggregation, which may lead to the loss of protein activity. Therefore, in some embodiments (and particularly in embodiments including a growth factor), the composition of the present invention includes a protein stabilizer. In some embodiments, the protein stabilizer forms a glassy phase around the growth factor, thereby preserving the protein structure. In some embodiments, the protein stabilizer is trehalose. Formulations including trehalose and methods of using trehalose in accordance with the present invention are disclosed in U.S. Provisional Patent Application Ser. No. 60/870,032, filed Dec. 14, 2006, entitled “Protein Stabilization Formulations” (Attorney Docket Number DEP-5877), the specification of which is incorporated by reference in its entirety.

Among the buffers, glycine was found to better protect rhGDF-5 in the trehalose formulation than trehalose alone.

### Sutures

It has been found that the compositions of the present invention can be suitably used on a wide variety of sutures. Specific sutures which have been demonstrated to provide suitable coating characteristics, drying times and solubility times with the compositions of the present invention include, but are not limited to, Orthocord-223104, Vicryl-J496, Plain Gut-844, Chronic Gut-S114, PDS II-Z347, and Ethibond Excel-X412.

In prophetically manufacturing the coated suture, lyophilized rhGDF-5 with trehalose protein stabilizer and glycine are dissolved in a SAIB solution. The suture is then dipped into this solution for a specific amount of time, and exposed to natural air-dry conditions for a specific amount of time to form a dry coat. The coated suture can then be used directly for clinical applications.

In one preferred prophetic method of manufacturing, a suitable amount of the SAIB carrier is dissolved in ethanol, n-methyl pyrrolidone (NMP), N-N-dimethyl formamide (DMF), dimethylsulfoxide (DMSO) or a mixture of at least two of these solvents in a volumetric ratio of 1:9 to 9:1. The concentration of the carrier is between about 2 wt % and 95 wt %. The carrier solution will be used to dissolve the rhGDF-5 and trehalose/glycine. The suture will be dipped into the drug/carrier solution contained within a reaction vessel for a specific amount of time, with the suture surface substantially completely in contact with the drug/carrier solution. The wet suture will be taken out of the reaction vessel and exposed for a few seconds to air for natural air drying. Health care professionals can then use this coated suture immediately for surgical procedures.

In another preferred prophetic method of manufacturing, a suitable amount of SAIB will be dissolved in N-methyl pyriolidone (NMP) and ethanol (EtOH) as the carrier in a sterile vial. The NMP and EtOH are used as solvents with the SAIB carrier. This carrier solution will be used to dissolve the contents of a second vial, which consists of lyophilized rhGDF-5 with trehalose. The trehalose is used for stabilizing the rhGDF-5 protein at 2-8° C. The suture will be dipped into this reconstituted rhGDF-5 SAIB solution for a specific amount of time (typically about 2 minutes), with the suture surface completely in contact with the rhGDF-5 SAIB solution. The wet suture will be taken out of the vial and air-dried for a specific amount of time at ambient temperature, usually taking a few seconds. Healthcare professionals can use this coated suture immediately for surgical procedures.

### Kits

In some embodiments, there is provided a kit for making a coated suture, comprising:
a) a first vial containing a growth factor (preferably, rhGDF-5) and a growth factor stabilizer (such as trehalose/glycine), and
b) a second vial containing:
   i) a biodegradable, non-polymeric, non-water soluble, liquid carrier material (such as SAIB),
   ii) a solvent in which both the biodegradable, non-polymeric, non-water soluble, liquid carrier material and the protein stabilizer are miscible (preferably, an ampiphilic solvent such as NMP), and
   iii) a volatile alcohol (such as ethanol).

In some embodiments, an rhGDF-5-coated suture having a biodegradable small-molecule carrier is used to enhance the healing of soft tissue repairs and improve healing in tendons, ligaments, and rotator cuff injuries. This includes rotator cuff tears, Achilles tendon rupture, flexor tendon tears, meniscal tears, and ACL reconstruction.

In some embodiments, the present invention is provided in a product package containing:
a) a package with a sterile suture,
b) a sterile “lyophilized protein” vial of rhGDF-5 with trehalose/glycine, at a dose of 0.5 mg or 1 mg of rhGDF-5/vial (which can be stored at 2-8° C. conditions), and
c) a sterile vial of SAIB with NMP and EtOH “carrier solution” (which can be stored at 2-8° C. or at room temperature conditions, and
d) instructions of how to use this product.

The first vial contains 0.5 to 2 mg lyophilized rhGDF-5, 50 mg trehalose and 0.375 mg glycine. The second vial contains 1.5 ml of liquid and has 10%-75% SAIB, with the balance being NMP: ethanol in a ratio of between 1:3 and 3:1.

In one embodiment, the instructions for using the product are as follows: First, tear off the seal of liquid “carrier solution” vial, swipe the stopper surface with alcohol swab. Carefully withdraw 1 ml of “carrier solution” using sterile syringe fitted with needle. Tear off the seal of “lyophilized protein” vial, swipe the stopper surface with alcohol swab. Carefully inject 1 ml “carrier solution”, which was withdrawn in step 2, into the “lyophilized protein” vial and gently swirl the vial for few seconds. Let the lyophilized cake become completely reconstituted, which generally takes 2 to 3 minutes. Uncap the vial after the cake is completely soluble. Soak the sterile suture in the vial of step 6 for about 30 seconds. Take out the soaked suture, let it dry for at least 15 to 30 seconds before using. Apply coated suture at surgical site.

### EXAMPLES

### Experiment 1

Initial experiments were done to understand the solubility of the various combinations of the reagents mentioned above. The following compositions were tested:
specific solvent composition:

| | |
|---|---|
| EtOH:NMP (1:3) > | 3.3 ml EtOH and 10 ml NMP |
| EtOH:NMP (1:1) > | 10 ml EtOH and 10 ml NMP |
| 10% SAIB > | 1.0 g SAIB with 10 ml EtOH and 10 ml NMP |
| 25% SAIB > | 2.5 g SAIB with 10 ml EtOH and 10 ml NMP |
| 50% SAIB > | 5.0 g SAIB with 3.3 ml EtOH and 10 ml NMP |
| EtOH:H₂O (1:1) > | 5 ml EtOH and 5 ml H₂0 |
| EtOH:H₂O (1:1) & | 5 ml EtOH and 5 ml H₂0 & 1.0 g SAIB with |
| 10% SAIB > | 10 ml EtOH and 10 ml NMP |

Solubility times ranged from ˜20-30 seconds to 600 seconds (5 minutes). The 25% SAIB solution had the longest solubility time of 600 seconds (5 minutes). The drying times for most of the sutures lasted for seconds, rather than minutes. Further experiments investigated the interactions between six different suture types (mentioned above) and various trehalose/glycine formulations to determine the drying times. The fastest drying times were ˜5-10 seconds.

Increasing ratios of EtOH:NMP has a minimal/no effect on the drying time of the Orthocord suture. Also, there is no distinct pattern that shows an increase in EtOH:NMP effects solubility time, as the data points with the 25% SAIB and 50% SAIB clearly had the higher solubility time points, suggesting SAIB may increase the solubility time on the Orthocord suture.

### Experiment 2

SEM data was generated for three suture types to understand how coverage of a single coat of various trehalose formulations affects uniformity and consistency on multi-filament sutures (VICRYL and Ethibond Excel) and mono-filament sutures (PDS II).

The following solvents were used:

| | |
|---|---|
| EtOH:NMP (1:3) > | 3.3 ml EtOH and 10 ml NMP |
| 25% SAIB > | 2.5 g SAIB with 10 ml EtOH and 10 ml NMP |
| EtOH:H2O & | 5 ml EtOH and 5 ml H₂0 & 1.5 g SAIB with |
| 10% SAIB > | 10 ml EtOH and 10 ml NMP |

There did not appear to be pronounced differences between the fiber coatings. The SEM data of Vicryl does not show any significant coating differences between the different solutions used. The EtOH:H₂O & 10% SAIB formulation showed a more consistent coat though, at 150× magnification. The SEM data of PDS II does not show any significant coating differences between the different solutions used, at 150× magnification. The SEM data of Ethibond Excel does not show any significant coating differences between the different solutions used, at 150× magnification. The EtOH:H₂O & 10% SAIB formulation showed a more rough coat though, at 150× magnification.

### Experiment 3

Another series of experiments was performed with Orthocord, Ethibond Excel and PDS II sutures. Sutures were coated multiple times (0, 1, 2, 3×) in a solvent mixture with trehalose/glycine (dip coating for 5 seconds and intervals between dip-coating were 5 seconds). The solvent mixture was EtOH:H₂O & 10% SAIB (5 ml EtOH and 5 ml H₂O & 1.5 g SAIB with 10 ml EtOH and 10 ml NMP).

SEM results did not show any substantial differences between multiple dip-coats onto sutures. There were some crystallized particles from the formulations present on the suture surfaces. Potentially, these particles could come off during suturing into and through repair tissue. The multiple dip coat suture test did not show any substantial differences under SEM at 100× magnification. The suture drying times for Orthocord and Ethibond Excel increased with multiple dip coats. The suture drying time for PDS II remained the same with multiple dip coats.

### Experiment 4

Experiments were conducted to assess whether increased concentrations of SAIB had any affect on Suture Dip Coat Testing. Orthocord sutures were coated (each suture 2 times) and then air dried vertically. SAIB concentrations were increased from 25% to 50% and 75% (with and without trehalose/glycine).

The following solvents were used:

| | |
|---|---|
| 25% SAIB > | 2.5 g SAIB with 5 ml EtOH and 5 ml NMP |
| 50% SAIB > | 5.0 g SAIB with 5 ml EtOH and 5 ml NMP |
| 75% SAIB > | 7.5 g SAIB with 5 ml EtOH and 5 ml NMP. |

SEM results at 100× showed the trehalose/glycine coated suture had a fine layer covering the suture while the uncoated suture had no layer.
This experiment showed that increasing SAIB from 25% to 50% to 75% (with and without trehalose) causes an increase in the drying time for the Orthocord suture. Also, the drying time with trehalose is lower than without trehalose/glycine. The data suggests that drying time increases with increasing SAIB concentrations.

### Experiment 5

This experiment showed the effect of coating the sutures with rhGDF-5 protein and the in vitro release of rhGDF-5 from the coated sutures in a buffer at 37° C.

Orthocord suture (original length of 36″) was cut to 12″ for this study, and dip coated with 1 ml of 1 mg rhGDF-5/50 mg trehalose/0.375 mg glycine solution 3 times, totaling 1 minute. The protein/trehalose/glycine cake was dissolved in 1 ml of 50% SAIB in 1:1 EtOH:NMP. The dry time was 80 seconds, and then this suture was immersed in 10 ml phosphate buffer at 37° C.

This buffer solution was refreshed according to the following schedule, and the protein concentration was determined using ELISA. The following results show a cumulative release of protein from the suture:

| Time (days) | Cumulative Protein concentration (ng) |
|---|---|
| 0 | 90.26 |
| 6 | 110 |
| 10 | 140.35 |
| 15 | 166.07 |
| 20 | 167.23 |

These results show that the protein is being released in a controlled fashion for an extended period of time.

We claim:

1. A surgical suture having a coating thereon comprising an effective amount of a growth factor and a biodegradable, non-polymeric, non-water soluble, liquid carrier.

2. The suture of claim 1 wherein the growth factor is a BMP.

3. The suture of claim 2 wherein the BMP is rhGDF-5.

4. The suture of claim 1 wherein the coating further comprises an ampiphilic solvent.

5. The suture of claim 4 wherein the ampiphilic solvent is NMP.

6. The suture of claim 1 wherein the coating further comprises an alcohol.

7. The suture of claim 6 wherein the alcohol is ethanol.

8. The suture of claim 1 wherein the coating further comprises a protein stabilizer.

9. The suture of claim 8 wherein the protein stabilizer is trehalose.

10. The suture of claim 1 wherein the carrier is selected from the group consisting of sucrose acetate isobutyrate (SAIB), sucrose acetate, sucrose octa acetate, dioctyladipate, medium and long chain fatty acid esters with 10-24 carbon atoms, medium and long chain phospholipids with 10-24 carbon atoms, medium and long chain diglycerides with 10-24 carbon atoms, medium and long chain triglycerides with 10-24 carbon atoms, butyl phthalate esters, sterol esters, steroid esters and vitamin E esters.

11. The suture of claim 1 wherein the carrier is sucrose acetate isobutyrate (SAIB).

12. The suture of claim 11 wherein the growth factor is a BMP.

13. The suture of claim 12 wherein the BMP is rhGDF-5.

14. A surgical suture having a coating thereon comprising an effective amount of:
a) a biologically active substance and
b) a biodegradable, non-polymeric, non-water soluble, liquid carrier material.

15. The suture according to claim 14, wherein the liquid carrier material is sucrose acetate isobutyrate.

16. The suture according to claim 14, wherein the carrier material has a viscosity of at least 10,000 cP at 37° C.

17. The suture according to claim 16, wherein the carrier material has a viscosity of at least 15,000 cP at 37° C.

18. The suture according to claim 17, wherein the carrier material has a viscosity of at least 20,000 cP at 37° C.

19. The suture according to claim 18, wherein the carrier material has a viscosity of at least 25,000 cP at 37° C.

20. The suture according to claim 19, wherein the carrier material has a viscosity of at least 50,000 cP at 37° C.

21. The suture according to claim 15, wherein the carrier material is present in an amount from about 95 percent to about 1 percent by weight, relative to the total weight of the coating.

22. The suture according to claim 15, wherein the carrier material is present in an amount from about 90 percent to about 5 percent by weight, relative to the total weight of the coating.

23. The suture of claim 15, wherein the carrier material is present in an amount from about 90 percent to about 10 percent by weight, relative to the total weight of the coating.

24. The composition of claim 14, wherein the carrier is a disaccharide acetate butyrate.

25. The composition of claim 14, wherein the carrier is a disaccharide ester.

26. The suture according to claim 14, wherein the coating further comprises an additive.

27. The suture according to claim 26, wherein the additive is selected from the group consisting of biodegradable polymers, non-biodegradable polymers, natural oils, synthetic oils, carbohydrates, carbohydrate derivatives, inorganic salts, and inert organic compounds.

28. The suture of claim 26, wherein the additive is selected from group consisting of a poly(lactide), a poly(lactide-co-glycolide), a poly(glycolide), poly(caprolactone), is oxidized cellulose, poly(D,L-lactic acid), a nonbiodegradable polymer, a biodegradable polymer, polyvinyl pyrrolidone, polyethylene glycol, cellulose acetate butyrate, cellulose acetate propionate, peanut oil, sesame oil,sucrose,doxycycline, and a carbohydrate.

29. The suture of claim 15, wherein the coating further comprises a solvent in which the non-polymeric non-water insoluble liquid carrier material is soluble.

30. The suture of claim 29, wherein the solvent is selected from the group consisting of n-methyl pyrrolidone (NMP), N-N-dimethyl formamide (DMF), and dimethylsulfoxide (DMSO).

31. The suture of claim 29, wherein the solvent is present in an amount from about 10 to about 50 percent by weight, relative to the weight of the composition.

32. The suture of claim 29, wherein the solvent is N-methyl pyrrolidone.

33. The suture of claim 15, wherein the coating further comprises a lower viscosity liquid carrier material.

34. The suture of claim 33, wherein the lower viscosity liquid carrier material has a viscosity of less than 1000 cp.

35. The suture of claim 15, wherein the biologically active substance is selected from the group consisting of peptides, proteins, nucleoproteins, mucoproteins, lipoproteins, and synthetic polypeptides.

36. The suture of claim 14, wherein the biologically active substance is a peptide.

37. The suture of claim 14, wherein the biologically active substance is a growth factor.

38. The suture of claim 37 wherein the growth factor is a member of a TGF beta superfamily.

39. The suture of claim 37 wherein the growth factor is a BMP.

40. The suture of claim 39 wherein the BMP is rhGDF-5.

41. The suture of claim 14 wherein the carrier material comprises a nonpolymeric ester or mixed ester of one or more carboxylic acids.

42. The suture of claim 41 wherein the ester is formed from a carboxylic acid that is esterified with a polyol having from about 2 to about 20 hydroxy moieties.

43. The suture of claim 41 wherein the ester further comprises 1 to about 20 etherified polyols.

44. The suture of claim 42 wherein the carboxylic acid has a hydroxy group obtained by ring opening alcoholysis of lactones, or cyclic carbonates or by the alcoholysis of carboxylic acid anhydrides.

45. The suture of claim 41 wherein the ester is formed from an amino acid that is esterified with a polyol having from about 2 to about 20 hydroxy moieties.

46. The suture of claim 41 wherein the esterified carboxylic acid is selected from the group consisting of glycolic acid, lactic acid, ε-hydroxycaproic acid, serine, and any corresponding lactones or lactams, trimethylene carbonate, and dioxanone.

47. The suture of claim 41 wherein an alcohol moiety of the ester or mixed ester is derived from a polyhydroxy alcohol having from about 2 to about 20 hydroxy groups.

48. The suture of claim 47 wherein the alcohol moiety is derived by removing one or more hydrogen atoms from a compound selected from the group consisting of: monofunctional C₁-C₂₀ alcohols, difunctional C₁-C₂₀ alcohols, trifunctional alcohols, hydroxy-containing carboxylic acids, hydroxy-containing amino acids, phosphate-containing alcohols, tetrafunctional alcohols, sugar alcohols, monosaccharides, and disaccharides, sugar acids, and polyether polyols.

49. The suture of claim 41 wherein each of the carboxylic acid moieties comprise at least one oxy moiety.

50. The suture of claim 41 wherein at least one of the carboxylic acid moieties of the ester or mixed ester contains 2 to 4 carbon atoms.

51. The suture of claim 50 wherein at least one of the carboxylic acid moieties further comprises at least one oxy moiety.

52. A suture coated with a composition comprising:
a) a growth factor,
b) a biodegradable, non-polymeric, non-water soluble, liquid carrier material,
c) a growth factor stabilizer,
d) a solvent in which both the biodegradable, non-polymeric, non-water soluble, liquid carrier material and the protein stabilizer are miscible, and
e) a volatile alcohol.

53. The suture of claim 52 wherein the growth factor is rhGDF-5.

54. The suture of claim 53 wherein the biodegradable, non-polymeric, non-water soluble, liquid carrier material is SAIB.

55. The suture of claim 54 wherein the growth factor stabilizer is trehalose.

56. The suture of claim 55 wherein the solvent is an ampiphilic solvent.

57. The suture of claim 56 wherein the volatile alcohol is ethanol.

58. A suture coated with a composition comprising:
a) a biodegradable, non-polymeric, non-water soluble, liquid carrier material,
b) a growth factor stabilizer, and
c) an ampiphilic solvent,

59. The suture of claim 58 wherein the carrier material is SAIB.

60. The suture of claim 59 wherein the stabilizer is trehalose.

61. The suture of claim 60 wherein the ampiphilic solvent is NMP.

62. A suture coated with a composition comprising:
a) an ampiphilic solvent, and
b) a volatile alcohol.

63. The suture of claim 62 wherein the ampiphilic solvent is NMP.

64. The suture of claim 63 wherein the alcohol is ethanol.

65. A kit for making a coated suture, comprising:
a) a first vial containing a growth factor and a growth factor stabilizer, and
b) a second vial containing:
i) a biodegradable, non-polymeric, non-water soluble, liquid carrier material,
ii) a solvent in which both the biodegradable, non-polymeric, non-water soluble, liquid carrier material and the protein stabilizer are miscible, and
iii) a volatile alcohol.

66. The kit of claim 65 wherein the growth factor is rhGDF-5.

67. The kit of claim 66 wherein the biodegradable, non-polymeric, non-water soluble, liquid carrier material is SAIB.

68. The kit of claim 67 wherein the solvent is an ampiphilic solvent.

69. The kit of claim 68 wherein the stabilizer is trehalose.

70. The kit of claim 69 wherein the alcohol is ethanol.
